(19) United States
(12) Patent Application Publication
(10) Pub. No.: US 2017/0260186 A1
(43) Pub. Date: Sep. 14, 2017

(54) PROCESS OF PREPARING AMORPHOUS FORM OF IDELALISIB

(71) Applicant: ScinoPharm Taiwan, Ltd., Shan-Hua (TW)

(72) Inventors: Tsung-Cheng Hu, Shan-Hua (TW); Wei-Shuo Lo, Shan-Hua (TW); Kuan-Hsun Wang, Shan-Hua (TW); Wan-Yin Cheng, Shan-Hua (TW)

(21) Appl. No.: 15/446,190

(22) Filed: Mar. 1, 2017

Related U.S. Application Data
(60) Provisional application No. 62/305,592, filed on Mar. 9, 2016.

(51) Int. Cl. C07D 473/34

(52) U.S. Cl. CPC C07D 473/34

(57) ABSTRACT

A process for preparing an amorphous form of idelalisib comprises:
(a) forming a first solution of idelalisib in a solvent;
(b) adding the first solution obtained in step (a) into water to form a second solution;
(c) optionally cooling the second solution obtained in step (b) and;
(d) isolating the optionally cooled second solution from step (c) to provide the amorphous form of idelalisib. Mild reaction conditions are used in this process. The amorphous form of idelalisib prepared by this process is stable and does not transfer to other crystalline forms easily. This process is suitable for large scale manufacture of idelalisib at a high yield.

### RELATED APPLICATIONS

This application claims priority from U.S. Provisional Patent Application Ser. No. 62/305,592, which was filed on Mar. 9, 2016. The entire content of these two provisional applications is incorporated herein as reference.

### STATEMENT AS TO RIGHTS TO INVENTIONS MADE UNDER FEDERALLY SPONSORED RESEARCH AND DEVELOPMENT

NOT APPLICABLE.

### REFERENCE TO A “SEQUENCE LISTING” A TABLE, OR A COMPUTER PROGRAM LISTING APPENDIX SUBMITTED ON A COMPACT DISK

NOT APPLICABLE.

### BACKGROUND OF THE INVENTION

The present invention relates to a process for preparing amorphous form of idelalisib which has the chemical name (S)-2-(1-(9H-purin-6-ylamino)propyl)-5-fluoro-3-phenylquinazolin-4(3H)-one and is represented by the structure:

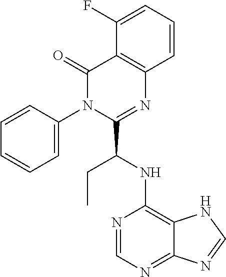 [SMILES: CC[C@H](Nc1ncnc2nc[nH]c12)c1nc2cccc(F)c2c(=O)n1-c1ccccc1]

Idelalisib (marketed as Zydelig®, codenamed GS-1101 or CAL-101) is a drug used for the treatment of chronic lymphocytic leukemia and was approved by the U.S. Food and Drug Administration (FDA) on Jul. 23, 2014.

U.S. Pat. No. 8,865,730 B2 discloses various forms of idelalisib, Form I (anhydrous), Form II (anhydrous), Form III (IPA/water solvate), Form IV (DMF solvate), Form V (DMSO solvate), Form VI (DCM solvate) and Form VII (water/ethanol solvate). International (PCT) Publication No. WO 2015092810A2 discloses an amorphous form of idelalisib by providing a solution or suspension of idelalisib in one or more solvents and obtaining the amorphous form of idelalisib by the removal of the solvent.

In view of the above, the present invention provides a process which can effectively afford amorphous idelalisib for drug development.

### SUMMARY OF THE INVENTION

The present invention provides an amorphous form of idelalisib and the preparation method thereof. In one aspect, the present invention provides a process for preparing the amorphous form of idelalisib, the process comprising:
(a) providing a first solution of idelalisib in a solvent;
(b) adding the first solution obtained in step (a) into water to form a second solution;
(c) optionally cooling the solution obtained in step (b) and;
(d) isolating the optionally cooled second solution to provide the amorphous form of idelalisib.

### BRIEF DESCRIPTION OF THE DRAWINGS

FIG. 1 shows the XRPD pattern for the amorphous form of idelalisib by MeOH/PPW system.

FIG. 2 shows the TGA thermogram for the amorphous form of idelalisib.

FIG. 3 shows the DSC thermogram for the amorphous form of idelalisib.

FIG. 4 shows the DVS isotherm plot for the amorphous form of idelalisib.

FIG. 5 shows the XRPD pattern for the amorphous form of idelalisib by DMSO/PPW system.

### DETAILED DESCRIPTION OF PREFERRED EMBODIMENTS OF THE INVENTION

The processes provided herein for the preparation of the amorphous form of idelalisib, have the benefits using mild reaction conditions. The amorphous form of idelalisib prepared by the present invention is stable and would not transfer to other crystalline form easily. The present invention provides a novel process for preparing amorphous form of idelalisib with high yield, and this method is suitable for the manufacture of idelalisib for large scale.

In one aspect, the present invention provides a process for preparing the amorphous form of idelalisib, the process comprising:
(a) forming a solution of idelalisib in a solvent;
(b) adding the solution obtained in step (a) into water;
(c) optionally cooling the solution obtained in step (b); and
(d) isolating the solution from step (c) to provide amorphous form of idelalisib.

In some embodiments, the solvent of step (a) is selected from methanol or dimethyl sulfoxide. In some embodiments, the cooling temperature of step (b) is below 10° C. In some embodiments, the cooling temperature is at 5° C.

In a related aspect, the present invention provides a process for preparing the amorphous form of idelalisib, the process comprising:
(a) forming a solution of idelalisib in a solvent;
(b) adding the solution obtained in step (a) into water;
(c) isolating the solution from step (b) to provide amorphous form of idelalisib.

In some embodiments, the solvent of step (a) is dimethyl sulfoxide. In some embodiments, the temperature of the solution of step (a) is at 25° C.

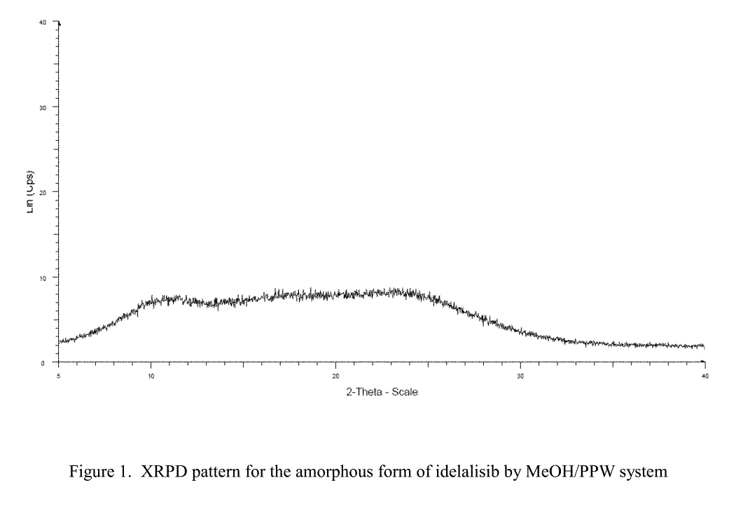

In some embodiments, the amorphous form of idelalisib by using methanol as solvent is characterized by an XRPD pattern with no significant peaks as provided in FIG. 1.

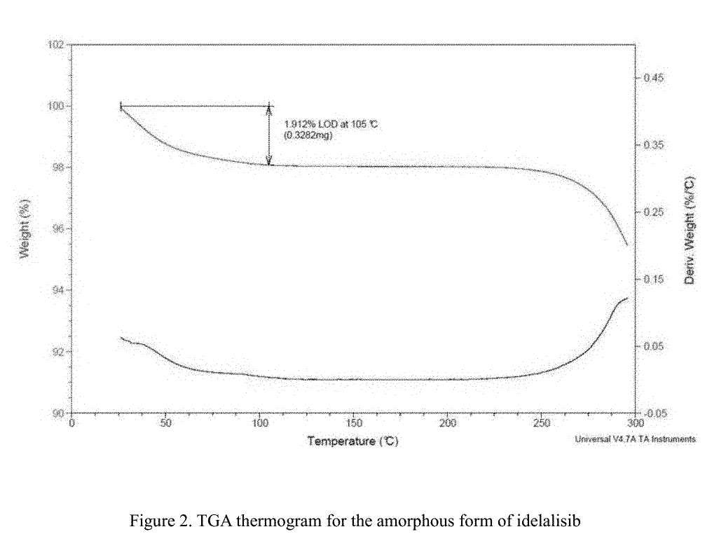

The thermal analysis of the amorphous form of idelalisib was conducted by TGA and DSC. In TGA thermogram FIG. 2, a weight loss occurred with temperature elevated and about 1.91% of weight loss was obtained at 105° C. followed by a sharp weight loss as the temperature was up to ˜250° C. The weight loss obtained from TGA curve is greater than the finding from water content analysis (0.61%) due to the product's hygroscopic nature. The TGA analysis was conducted in laboratory environment without humidity controlled.

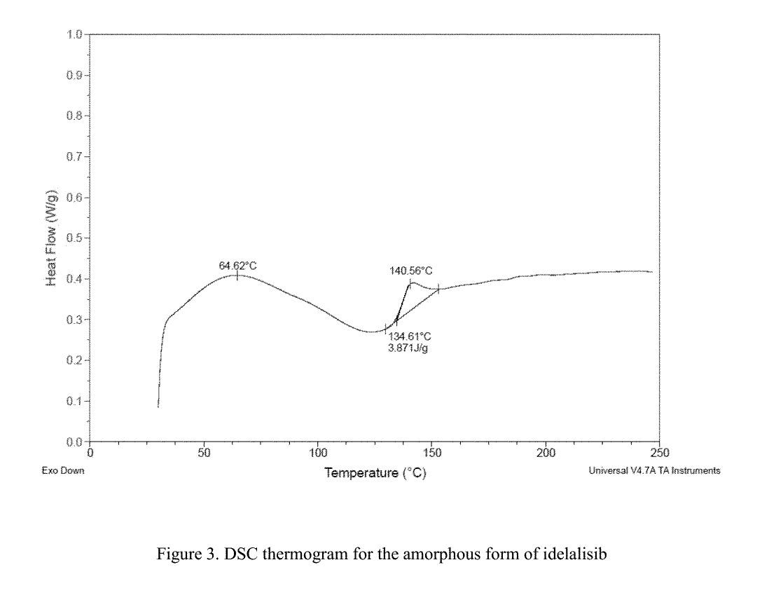

In DSC thermogram FIG. 3, two endothermic peaks with maximum temperatures at 64.6 and 140.6° C. were observed.

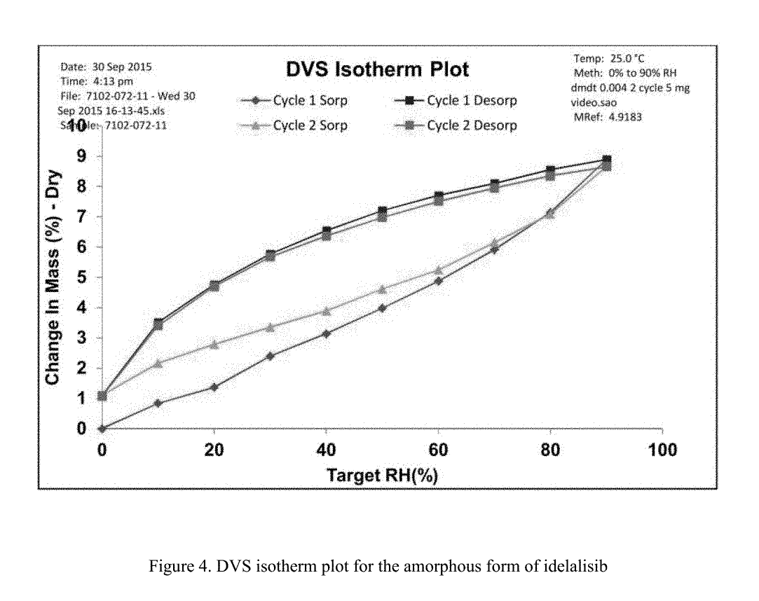

The water-solid interaction investigated of amorphous idelalisib using Dynamic Vapor Sorption (DVS) analyzer shows that approximately 8.9% moisture uptake is obtained when the sample is exposed in the humidity condition changing from 0% RH to 90% RH. The DVS isotherm plot of amorphous idelalisib is shown in FIG. 4. The DVS results indicated amorphous Idelalisib is a hygroscopic material and should be well packed and stored with protecting from excessive moisture. It is recommended to handle the sample in a controlled environment with humidity <10% RH to avoid any unnecessary moisture uptake.

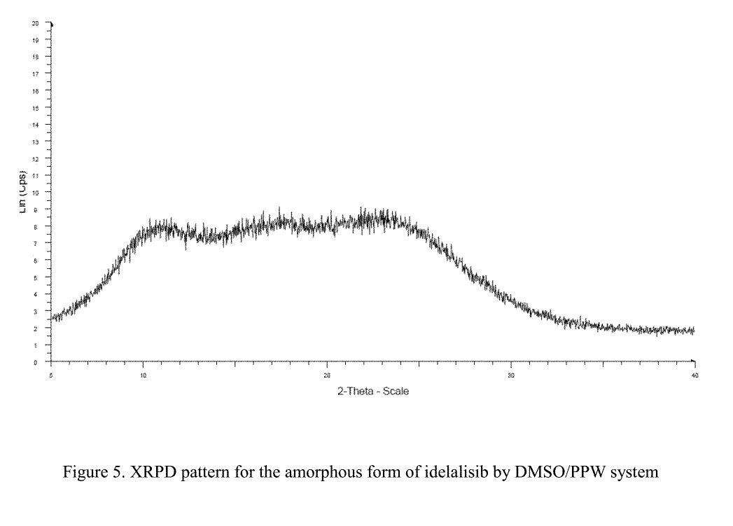

In some embodiments, the amorphous form of idelalisib by using dimethyl sulfoxide as solvent is characterized by an XRPD pattern with no significant peaks as provided in FIG. 5.

The preliminary physicochemical stability of the amorphous idelalisib was studied under the recommended long term storage condition (25° C./60% RH) and accelerated condition (40° C./75% RH) for 3 months. The amorphous idelalisib was recommended to be packed and stored following the procedures below: wrap amorphous idelalisib inside a Low-Density PolyEthylene (LDPE) bag under the humidity controlled environment with a relative humidity lower than 10% and again wrap inside a second LDPE bag. The double packed LDPE bag was then placed inside an aluminum foil bag with appropriate amount of desiccant between the LDPE bag and the aluminum foil bag. The aluminum bag package was then placed in a High-Density PolyEthylene (HDPE) container and stored at temperature below 25° C. There was no significant degradation impurity detected, the water content was not increased and the solid form remained as amorphous under storage condition (25° C./60% RH) and accelerated condition (40° C./75% RH) for 3 months. The results indicate that the recommended packaging and storage condition described above are compatible to amorphous idelalisib.

Powder X-Ray Diffractometer (PXRD)

X-ray Powder Diffraction patterns were collected on a Bruker AXS D8 Advance diffractometer using Cu Kα₁ radiation (40 kV, 40 mA), 0-20 goniometer, and divergence of V4 and receiving slits, a Ge monochromator and LynxEye detector. The representative XRPD pattern was collected under ambient condition. The details of the scanning parameters are:
Angular range: 5-40°
Step size: 0.02°
Scan speed: 0.6 sec/step

Thermo Gravimetric Analysis (TGA)

TGA data was collected on a TA instrument Q500 TGA. Each sample (15-20 mg) was loaded onto a pre-tared platinum crucible; the balance and furnace were purged with nitrogen prior to the analysis with a flow rate set as 40±5 and 60±5 mL/min, respectively. The heating process was programmed to start at the ambient temperature and stop at 300° C. with a 10° C./min ramp.

Differential Scanning Calorimetry (DSC)

DSC data was collected on a TA Instrument MDSC Q200. Each sample (2-5 mg) was loaded onto a hermetic pan with pinhole and the analysis was carried out under a constant flow of nitrogen (60 mL/min). The heating process was programmed to start from 30° C. and stop at 250° C. with a 10° C./min ramp.

Dynamic Vapor Sorption (DVS)

The sample was placed into the DVS sample pan and dried under a stream of dry nitrogen at 25° C. with 0% RH. The moisture was gradually introduced into the system with a 10% RH increment up to 90% RH and the humidity was then decreased in a similar trend for desorption phase. The sorption and desorption data were collected with equilibration set to dm/dt 0.004%/min for 5 min/step. The minimum and maximum time for each step were set to 10 and 360 min. Two sorption/desorption cycles were performed.

### EXAMPLES

The following examples are provided to further illustrate, but not to limit this invention.

### Example 1

Idelalisib (1.005 g, 1.0 equiv) and MeOH (10 mL, 10 vol) were charged into a suitable vessel. The mixture was stirred at 50° C. until a homogeneous solution was achieved. After dissolved, the hot resulting solution was added into PPW (100 mL, 100 vol) for about 5 min at RT, and then stirred for NLT 0.5 hr. The solids were filtered by vacuum suction, purged with nitrogen for NLT 1 hr, and vacuum drying (about 100 torr) at NMT 60° C. to afford 0.890 g of amorphous idelalisib with white to off white solid.

### Example 2

Idelalisib (10.03 g, 1.0 equiv) and MeOH (90 mL, 9 vol) were charged into a suitable vessel. The mixture was stirred at ˜55° C. to achieve a homogeneous solution. The resulting solution was cooled to room temperature (20 to 35° C.) and filtered followed by washed with MeOH (10 mL, 1 vol) twice through filter paper and added into PPW (800 mL, 80 vol) for NLT 0.5 hr at 25° C., and then stirred for NLT 1 hr. The resulting mixture was cooled to 0-5° C. and then stirred for NLT 1 hr. Afterward, the solids were filtered by vacuum suction and washed with a MeOH/PPW co-solution (v/v=1/8, 50 mL, 5 vol) twice. The wet cake was purged with nitrogen for NLT 1 hr, and vacuum drying (about 100 torr) at NMT 60° C. to afford amorphous idelalisib with white to off white solid.

### Example 3

Idelalisib (10.004 g, 1.0 equiv) and MeOH (90 mL, 9 vol) were charged into a suitable vessel. The mixture was stirred at 60˜65° C. to achieve a homogeneous solution. The resulting solution was cooled to room temperature (20 to 35° C.) and filtered followed by washed with MeOH (10 mL, 1 vol) through filter paper and added into PPW (400 mL, 40 vol) for NLT 0.5 hr at 25° C., and then stirred for NLT 1 hr. The resulting mixture was cooled to 0-5° C. and then stirred for NLT 1 hr. Afterward, the solids were filtered by vacuum suction and washed with a pre-cooled MeOH/PPW co-solution (v/v=1/4, 50 mL, 5 vol) twice. The wet cake was purged with nitrogen for NLT 1 hr, and vacuum drying (about 100 torr) at NMT 60° C. to afford 9.170 g of SPT1384API with white to off white solid.

### Example 4

Idelalisib in MeOH solution (˜10 g dissolved in 5 vol MeOH, 1.0 equiv) and MeOH (30-40 mL, 3-4 vol) were charged into a suitable vessel. The mixture was stirred at room temperature (20 to 35° C.) to achieve a homogeneous solution. The resulting solution was filtered followed by washed with MeOH (10 mL, 1 vol) through filter paper and added into PPW (400 mL, 40 vol) for NLT 0.5 hr at 25° C., and then stirred for NLT 1 hr. The resulting mixture was cooled to 0-5° C. and then stirred for NLT 1 hr. Afterward, the solids were filtered by vacuum suction and washed with a pre-cooled MeOH/PPW co-solution (v/v=1/4, 50 mL, 5 vol) twice. The wet cake was purged with nitrogen for NLT 1 hr, and vacuum drying (about 100 torr) at NMT 60° C. to afford 7.803 g of amorphous idelalisib with white to off white solid.

### Example 5

Idelalisib (0.503 g, 1.0 equiv) and DMSO (2 mL, 4 vol) were charged into a suitable vessel. The mixture was stirred at RT to achieve a homogeneous solution. The resulting solution was added into PPW (25 mL, 50 vol) at 25° C., and then stirred for NLT 1 hr. Afterward, the solids were filtered by vacuum suction and washed with PPW (5 mL, 10 vol) for five times. The wet cake was purged with nitrogen for NLT 1 hr, and vacuum drying (about 150 torr) at NMT 60° C. to afford amorphous idelalisib with white to off white solid.

### Example 6

Idelalisib (1.02 g, 1.0 equiv) and DMSO (4 mL, 4 vol) were charged into a suitable vessel. The mixture was stirred at RT to achieve a homogeneous solution. The resulting solution was added into PPW (50 mL, 50 vol) at 25° C., rinse with DMSO (2 mL, 2 vol), and then stirred for NLT 1 hr. Afterward, the solids were filtered by vacuum suction and washed with PPW (5 mL, 10 vol). The wet cake was purged with nitrogen for NLT 1 hr to afford 0.916 g of amorphous idelalisib with white to off white solid.

What is claimed is:

1. A process for preparing an amorphous form of idelalisib comprising:
(a) forming a first solution of idelalisib in a solvent;
(b) adding the first solution obtained in step (a) into water to form a second solution;
(c) optionally cooling the second solution obtained in step (b); and
(d) isolating the second solution to provide the amorphous form of idelalisib.

2. A process according to claim 1, wherein the solvent is selected from the group consisting of methanol and dimethyl sulfoxide.

3. A process according to claim 1, wherein the cooling temperature is below 10° C.

4. A process according to claim 3, wherein the cooling temperature is at 5° C.

5. A process according to claim 1, wherein the amorphous from of idelalisib is characterized by an XRPD pattern as shown in FIG. 1.

6. A process according to claim 1, wherein the amorphous from of idelalisib is characterized by a TGA pattern as shown in FIG. 2.

7. A process according to claim 1, wherein the amorphous from of idelalisib is characterized by a DSC pattern as shown in FIG. 3.

8. A process according to claim 1, wherein the amorphous from of idelalisib is characterized by a DVS isotherm plot as shown in FIG. 4.

9. A process for preparing an amorphous form of idelalisib comprising:
(a) providing a solution of idelalisib in a solvent;
(b) adding the solution obtained in step (a) into water;
(c) isolating the solution from step (b) to provide the amorphous form of idelalisib.

10. A process according to claim 9, wherein the solvent is dimethyl sulfoxide.

11. A process according to claim 9, wherein the temperature of the solution of step (a) is at 25° C.

12. A process according claim 9, wherein the amorphous from of idelalisib is characterized by a XRPD pattern as shown in FIG. 5.

13. An amorphous form of idelalisib characterized by a XRPD pattern as shown in FIG. 1, a TGA pattern as shown in FIG. 2, a DSC pattern as shown in FIG. 3, and/or a DVS isotherm plot as shown in FIG. 4.

14. A pharmaceutical formulation comprising the amorphous form of idelalisib of claim 13, and a pharmaceutically acceptable carrier.
